(19) United States
(12) Patent Application Publication
(10) Pub. No.: US 2003/0157040 A1
(43) Pub. Date: Aug. 21, 2003

(54) Use of ectoine or ectoine derivatives in cosmetic formulations

(75) Inventors: Joachim Bunger, Darmstadt (DE); Hans-Jurgen Driller, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Merck Patent GmbH, Darmstadt (DE)

(21) Appl. No.: 10/355,149

(22) Filed: Jan. 31, 2003

Related U.S. Application Data
(62) Division of application No. 09/744,766, filed on Jan. 30, 2001.
Application No. 09/744,766 is a 371 of international application No. PCT/EP99/05237, filed on Jul. 22, 1999.

(30) Foreign Application Priority Data
Aug. 1, 1998 (DE) 19834816.9

(51) Int. Cl. A61K 7/42; A61K 38/04; A61K 38/05; A61K 31/551; A61K 31/4172

(52) U.S. Cl. 424/59; 514/18; 514/19; 514/218; 514/256; 514/400

(57) ABSTRACT

The invention relates to the use of one or more compounds selected from the compounds of formulas (Ia) and (Ib), their physiologically compatible salts and their stereoisomeric forms, where R¹, R², R³, R⁴ and n have the meanings given in claim no. 1, for the preparation of a cosmetic formulation. Said use is advantageous in protecting and stabilizing the nucleic acids of human skin cells.

The invention relates to the use of one or more compounds chosen from the compounds of the formulae Ia and Ib

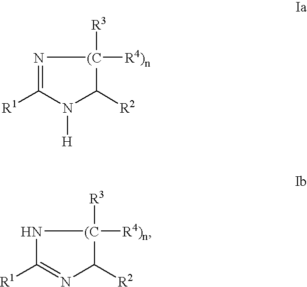

the physiologically compatible salts of the compounds of the formulae Ia and Ib, and the stereoisomeric forms of the compounds of the formulae Ia and Ib, where

R¹ is H or alkyl,

R² is H, COOH, COO-alkyl or CO—NH—R⁵,

R³ and R⁴ in each case independently of one another are H or OH,

n is 1, 2 or 3,

alkyl is an alkyl radical having 1 to 4 carbon atoms, and

R⁵ is H, alkyl, an amino acid radical, dipeptide radical or tripeptide radical for the preparation of a cosmetic formulation for the protection and stabilization of nucleic acids of human skin cells.

The invention relates in particular to the use of one or more compounds chosen from the compounds of the formulae Ia and Ib, the physiologically compatible salts of the compounds of the formulae Ia and Ib and the stereoisomeric forms of the compounds of the formulae Ia and Ib for the preparation of a cosmetic formulation for the protection and stabilization of the nucleic acids of human skin cells against physical, chemical and biological influences, such as e.g. against radiation (UV, VIS and IR radiation), in particular UV radiation, against denaturing substances, against enzymes, in particular endonucleases and restriction enzymes, and against viruses, in particular Herpes viruses.

The invention further relates, to the use of one or more compounds chosen from the compounds of the formulae Ia and Ib, the physiologically compatible salts of the compounds of formulae Ia and Ib and the stereoisomeric forms of the compounds of the formulae Ia and Ib together with one or more UV filters for the preparation of a cosmetic formulation for the protection and stabilization of the nucleic acids of human skin cells against UV radiation, in particular against UV-A radiation.

The nucleic acids, deoxyribonucleic acid (DNA) and ribonucleic acids (RNA), occur in all cells of the human body. They play a decisive role in heredity and control the formation of the proteins necessary for life and the function of every cell. Deoxyribonucleic acid carries the information for protein synthesis, while ribonucleic acids a-re involved in transferring this information to the protein synthesis. Damage to the nucleic acids of human skin cells, in particular to deoxyribonucleic acid, can lead to chronic changes in the skin.

Being the barrier layer and surface of the human body, the skin is exposed to a large number of external stress factors. The human skin is an organ which, having diverse specialized cell types—keratinocytes, melanocytes, Langerhans cells, Merkel cells and intercalated sensory cells—protects the body against external influences. It is necessary here to differentiate between external physical, chemical and biological influences on the human skin. External physical influences include thermal and mechanical influences, and the effect of radiation, e.g. UV, VIS and IR radiation. External chemical influences are, in particular, the effect of toxins, allergens and substances which bind to the deoxyribonucleic acid. External biological influences include the effect of foreign organisms and metabolic products thereof.

Sunlight has both positive and negative effects on the human skin and the entire organism. In a suitable dose, solar irradiation increases wellbeing and the performance of the organism. The synthesis of vitamin D is stimulated and, as a result of the irradiation, the desired tanning or pigmenting of the skin finally develops. The pigmenting is part of the intrinsic protection of the skin, which is based on a large number of mechanisms. In connection with the intrinsic protection of the skin, in addition to the pigmenting, the thickening of the horny layer (photocallosity), the dark repair system (enzymatic DNA repair), the redox systems for controlling free-radical reactions and the synthesis of urocanic acid are of importance (P. Finkel, “Lichtschutzmittel” [Light protection agents] in W. Umbach, Kosmetik, 2nd edition, 1995, 147-163, Georg Thieme Verlag, Stuttgart).

Excessive solar irradiation leads both to acute skin damage, such as, for example, sunburn, and also to chronic changes, such as, for example, skin aging or skin cancer. Sunburn (Erythema solare) develops predominantly as a result of UV-B irradiation. By contrast, UV-A radiation has a comparatively low influence on its development. Sunburn can occur from a slight reddening to severe burning with the formation of blisters. Since these consequences occur at the earliest 4-6 h following irradiation, it is too late for countermeasures. Sunburn is evidence or acute skin damage which may be of relevance for chronic changes in the skin. A number of instances of sunburn, especially in childhood, significantly increase the risk of skin cancer. Causes of this are damage, particularly to the nucleic acids of human skin cells, and defective repair of the damaged deoxyribonucleic acid in the cell nucleus, and presumably the immunosuppressive action of the UV radiation, i.e. the weakening of the immune reaction as a result of UV irradiation. Excessive UV-A and UV-B exposure contributes to skin aging or photoaging, e.g. in the form of structural changes in the connective tissue (actinic elastosis). Excessive UV-B exposure is the main cause of chronic changes in the skin.

Because of a change in leisure behavior, such as e.g. extensive sunbathing or travel to countries with high solar irradiation, the risks of UV damage to the skin cells have increased sharply in recent years, which in turn results in an increase in the risk of skin cancer (P. Finkel, “Lichtschutzmittel” [Light protection agents] in W. Umbach, Kosmetik, 2nd edition, 1995, 147-163, Georg Thieme Verlag, Stuttgart). A particular hazard potential is travel to countries with high solar irradiation in winter. The winter skin, e.g. of northern Europeans, is less pigmented and not protected against high solar exposure in tropical, equatorial regions with a long period of sunshine each day. In addition, the risk of skin cancer has risen significantly in recent times as a result of increased UV radiation on the surface of the earth, caused by the diminution of the ozone layer, and as a result of the higher life expectancy of the human race.

The object was therefore to provide cosmetic formulations, the use of which overcome [sic] or at least reduce [sic] the abovementioned skin problems and are [sic] suitable in particular for the protection and stabilization of nucleic acids of human skin cells.

In particular, the object was to provide cosmetic formulations which are suitable for the protection and stabilization of nucleic acids of human skin cells against physical, chemical and biological influences, such as e.g. against radiation (UV, VIS and IR radiation), in particular UV radiation, against denaturing substances, against enzymes, in particular endonucleases and restriction enzymes, and against viruses, in particular Herpes viruses.

Surprisingly, we have now found that this object is achieved by the use of one or more compounds chosen from the compounds of the formulae Ia and Ib

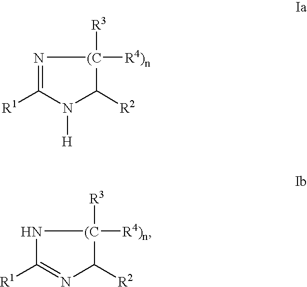

the physiologically compatible salts of the compounds of the formulae Ia and Ib, and the stereoisomeric forms of the compounds of the formulae Ia and Ib, where

R¹ is H or alkyl,

R² is H, COOH, COO-alkyl or CO—NH—R⁵,

R³ and R⁴ in each case independently of one another are H or OH,

n is 1, 2 or 3,

alkyl is an alkyl radical having 1 to 4 carbon atoms, and

R⁵ is H, alkyl, an amino acid radical, dipeptide radical or tripeptide radical

in cosmetic formulations.

In addition, we have found that the use of one or more compounds chosen from the compounds of the formulae Ia and Ib, the physiologically compatible salts of the compounds of the formulae Ia and Ib and the stereo-isomeric forms of the compounds of the formulae Ia and Ib together with one or more UV filters is suitable for the preparation of a cosmetic formulation for the protection and stabilization of the nucleic acids of human skin cells against UV radiation.

Within the scope of the present invention, all compounds above and below chosen from the compounds of the formulae Ia and Ib, the physiologically compatible salts of the compounds of the formulae Ia and Ib, and the stereoisomeric forms of the compounds of the formulae Ia and Ib are referred to as “ectoin or ectoin derivatives”.

The invention further relates to cosmetic formulations comprising

a) one or more compounds chosen from the compounds of the formulae Ia and Ib

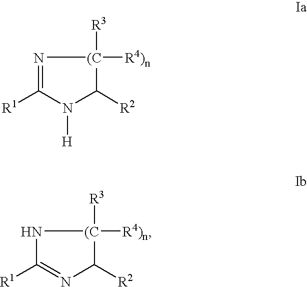

the physiologically compatible salts of the compounds of the formulae Ia and Ib, and the stereoisomeric forms of the compounds of the formulae Ia and Ib, where

R¹ is H or alkyl,

R² is H, COOH, COO-alkyl or CO—NH—R⁵,

R³ and R⁴ in each case independently of one another are H or OH,

n is 1, 2 or 3,

alkyl is an alkyl radical having 1 to 4 carbon atoms, and

R⁵ is H, alkyl, an amino acid radical, dipeptide radical or tripeptide radical and

b) one or more UV filters.

Cosmetic formulations which comprise ectoin or ectoin derivatives protect, in particular, deoxyribonucleic acid of human skin cells against physical stress, in particular against radiation, such as e.g. UV, VIS or IR radiation, and against chemical stress. In particular, formulations comprising ectoin or ectoin derivatives and sunscreen filters, in particular UV filters, offer excellent protection against UV-induced damage to the deoxyribonucleic acid of skin cells.

Following UV irradiation, individually stored keratinocytes having particular characteristics, the so-called “sunburn cells” (SBCs) form in the human epidermis. The formation of “sunburn cells” is described as one example of programmed cell death, apoptosis. The formation of “sunburn cells” with their characteristic pycnotic nucleus and the eosinophilic cytoplasm can be detected in the human epidermis even after moderate irradiation with UVC or UVB or UVA in combination with psoralen. The fate of an SBC, a keratinocyte which displays abnormal and very premature keratinization, is hitherto unknown. Which photo-chemical event leads to the formation of SBCs is likewise unknown. However, there is evidence that UV-induced DNA damage plays an important role in this process. It is, for example, suspected that the presence of SBCs could be a possible indicator of and pointer to a given photocarcinogenic potential. SBCs exhibit considerable vacuole formation in eosin-dyed cytoplasm, and also shrinkage with a considerably condensed cell nucleus. They detach from the cell aggregation and exhibit rapid, premature keratinization. They can be readily detected histologically in the prepared sections and can be evaluated quantitatively relative to the number of basal cells or the total number of keratinocytes. The action spectrum of the SBC formation is identical to that of the erythema. Photoaugmentation by UVA and psoralens (PUVA) likewise leads to a dose-dependant SBC formation [A. R. Young, ‘The sunburn cell’, Photodermatology 4 (1987) 127-134; G. Kindl et al., ‘Die Wirkung der Sonnenstrahlen’ in: Licht und Haut,[‘The effect of solar rays’ in: Light and Skin], Verlag Govi, Frankfurt am Main (1993) 51-69; H. Iizuka, ‘Effects of UVB irradiation on epidermal adenylate cyclase responses in vitro: its relation to Sunburn cell formation’, Arch. Dermatol. Res. 280 (1998) 163-167].

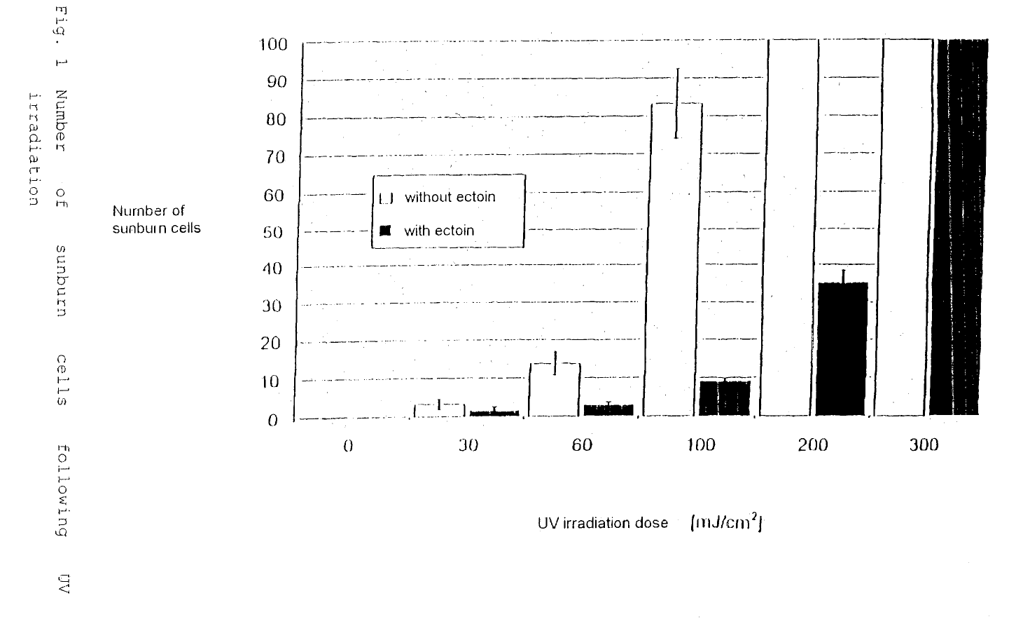

Within the scope of the present invention, ectoin was investigated with regard to its possible potential for reducing the UV-induced formation of SBCs. In order to be able to correctly analyze this potential of an active ingredient concept, it is necessary to study the appearance and the formation of SBCs in a physiological model which simulates the in vivo situation as closely as possible. This prerequisite is most likely to be achieved using an organotypical skin model for such studies. We were able to show that there is a dose-effect relationship between the rate of formation of SBCs and the administered UV dose. It was possible to shift this dose-effect relationship significantly into higher UV dose ranges by means of pretreatment with ectoin (see, FIG. 1).

Topically applied formulations which comprise ectoin or ectoin derivatives additionally protect nucleic acids against denaturing substances and enzymes, such as e.g. endonucleases and restriction enzymes. Ectoin or ectoin derivatives can also be used in cosmetic formulations against viruses, in particular Herpes viruses.

Ectoin and the ectoin derivatives are low molecular weight, cyclic amino acid derivatives which can be obtained from various halophilic microorganisms. Both ectoin and hydroxyectoin have the advantage that they do not react with the cell metabolism.

The compounds chosen from the compounds of the formulae Ia and Ib, the physiologically compatible salts of the compounds of the formulae Ia and Ib and the stereo-isomeric forms of the compounds of the formulae Ia and Ib may be present in the cosmetic preparations as optical isomers, diastereomers, racemates, zwitterions, cations or as a mixture thereof. Of the compounds chosen from the compounds of the formulae Ia and Ib, the physiologically compatible salts of the compounds of the formulae Ia and Ib and the stereoisomeric forms of the compounds of the formulae Ia and Ib, preference is given to those compounds in which R¹ is H or CH₃, R² is H or COOH, R³ and R⁴ are in each case independently of one another H or OH, and n is 2. Of the compounds chosen from the compounds of the formulae Ia and Ib, the physiologically compatible salts of the compounds of the formulae Ia and Ib and the stereoisomeric forms of the compounds of the formulae Ia and Ib, particular preference is given to the compounds (S)-1,4,5,6-tetrahydro-2-methyl-4-pyrimidine-carboxylic acid (ectoin) and (S,S)-1,4,5,6-tetrahydro-5-hydroxy-2-methyl-4-pyrimidinecarboxylic acid (hydroxyectoin).

The term “amino acids” means the stereoisomeric forms, e.g. D and L forms, of the following compounds: alanine, β-alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine, γ-aminobutyrate, Nε-acetyllysine, Nδ-acetylornithine, Nγ-acetyldiaminobutyrate and Nα-acetyldiaminobutyrate. L-Aminoacids are preferred.

Amino acid radicals are derived from the corresponding amino acids.

The radicals of the following amino acids are preferred: alanine, β-alanine, asparagine, aspartic acid, glutamine, glutamic acid, glycine, serine, threonine, valine, γ-aminobutyrate, Nε-acetyllysine, Nδ-acetylornithine, Nγ-acetyldiaminobutyrate and Nα-acetyldiaminobutyrate.

According to their chemical nature, the di- and tripeptide radicals are acid amides and decompose upon hydrolysis into 2 or 3 amino acids. The amino acids in the di- and tripeptide radicals are bonded together by amide bonds. Preferred di- and tripeptide radicals are made up of the preferred amino acids.

The alkyl groups include the methyl group CH₃, the ethyl group C₂H₅, the propyl groups CH₂CH₂CH₃ and CH(CH₃)₂, and the butyl groups CH₂CH₂CH₂CH₃, H₃CCHCH₂CH₃, CH₂CH(CH₃)₂ and C(CH₃)₃. The preferred alkyl group is the methyl group.

Preferred physiologically compatible salts of the compounds of the formulae Ia and Ib are, for example, alkali metal, alkaline earth metal or ammonium salts, such as Na, K, Mg or Ca salts, and salts derived from the organic bases triethylamine or tris(2-hydroxy-ethyl)amine. Further preferred physiologically compatible salts of the compounds of the formulae Ia and Ib arise by reaction with inorganic acids, such as hydrochloric acid, sulfuric acid and phosphoric acid, or with organic carboxylic or sulfonic acids, such as acetic acid, citric acid, benzoic acid, maleic acid, fumaric acid, tartaric acid and p-toluenesulfonic acid.

Compounds of the formulae Ia and Ib in which basic and acidic groups such as carboxyl or amino groups are present in equal number form internal salts.

The preparation of the compounds of the formulae Ia and Tb is described in the literature (DE 43 42 560). (S)-1,4,5,6-tetrahydro-2-methyl-4-pyrimidinecarboxylicic acid or (S,S)-1,4,5,6-tetrahydro-5-hydroxy-2-methyl-4-pyrimidinecarboxylic acid can also be obtained microbiologically (Severin et al., J. Gen. Microb. 138 (1992) 1629-1638).

For the use according to the invention of one or more compounds chosen from the formulae Ia and Ib, the physiologically compatible salts of the compounds of the formulae Ia and Ib and the stereoisomeric forms of the compounds of the formulae Ia and Ib together with one or more UV filters, and for the cosmetic formulation according to the invention comprising one or more UV filters, both organic and inorganic UV filters are suitable.

Suitable organic UV filters are e.g. benzoyl- or dibenzoylmethane derivatives, methoxycinnamates, salicylate derivatives, benzylidenecamphor derivatives, octocrylene, benzophenone, phenylbenzimidazole-5-sulfonic acid, 4-aminobenzoic acid, octyl triazone and octyl dimethyl PABA.

Suitable inorganic UV filters are e.g. titanium dioxide and zinc oxide.

According to the present invention, the use of one or more compounds chosen from the compounds of the formulae Ia and Ib, the physiologically compatible salts of the compounds of the formulae Ia and Ib and the stereoisomeric forms of the compounds of the formulae Ia and Ib, optionally together with one or more UV filters, for the preparation of a cosmetic formulation preferably leads to the protection and stabilization of the nucleic acids of the cells of the epidermis, in particular the keratinocytes, melanocytes, Langerhans cells and Merkel cells.

The cosmetic formulation is prepared by converting one or more compounds chosen from the compounds of the formulae Ia and Ib, the physiologically compatible salts of the compounds of the formulae Ia and Ib and the stereoisomeric forms of the compounds of the formulae Ia and Ib optionally together with one or more UV filters and optionally with auxiliaries and/or carrier substances into a suitable formulation form. The auxiliaries and carrier substances originate from the group of carriers, preservatives and other customary auxiliaries.

The cosmetic formulations based on one or more compounds chosen from the compounds of the formulae Ia and Ib, the physiologically compatible salts of the compounds of the formulae Ia and Ib and the stereoisomeric forms of the compounds of the formulae Ia and Ib and optionally additionally based on one or more UV filters are applied externally.

Examples of use forms which may be mentioned are: solutions, suspensions, emulsions, pastes, ointments, gels, creams, lotions, powders, soaps, surfactant-containing cleansing preparations, oils and sprays. In addition to one or more compounds chosen from the compounds of the formulae Is and Ib, the physiologically compatible salts of the compounds of the formulae Ia and Ib and the stereoisomeric forms of the compounds of the formulae Ia and Ib and optionally in addition to one or more UV filters, any customary carrier substances, auxiliaries and optionally further active ingredients are added to the formulation.

Preferred auxiliaries originate from the group of preservatives, antioxidants, stabilizers, solubility promoters, vitamins, coloring agents, odor improvers.

In addition to one or more compounds chosen from the compounds of the formulae Ia and Ib, the physiologically compatible salts of the compounds of the formulae Ia and Ib and the stereoisomeric forms of the compounds of the formulae Ia and Ib and optionally in addition to one or more UV filters, ointments, pastes, creams and gels can comprise the customary carrier substances, e.g. animal and vegetable fats, waxes, paraffins, starch, tragacanth, cellulose derivatives, polyethylene glycols, silicones, bentonites, silica, talc and zinc oxide or mixtures of these substances.

In addition to one or more compounds chosen from the compounds of the formulae Ia and Ib, the physiologically compatible salts of the compounds of the formulae Ia and Ib and the stereoisomeric forms of the compounds of the formulae Ia and Ib and optionally in addition to one or more UV filters, powders and sprays can comprise the customary carrier substances, e.g. lactose, talc, silica, aluminum hydroxide, calcium silicate and polyamide powder or mixtures of these substances. Sprays can additionally comprise the customary propellants, e.g. chlorofluorocarbons, propane/butane or dimethyl ether.

In addition to one or more compounds chosen from the compounds of the formulae Ia and Ib, the physiologically compatible salts of the compounds of the formulae Ia and Ib and the stereoisomeric forms of the compounds of the formulae Ia and Ib and optionally in addition to one or more UV filters, solutions and emulsions can comprise the customary carrier substances, such as solvents, solubility promoters and emulsifiers, e.g. water, ethanol, isopropanol, ethyl carbonate, ethyl acetate, benzyl alcohol, benzyl benzoate, propylene glycol, 1,3-diethylene glycol butyl ether, oils, in particular cottonseed oil, groundnut oil, wheatgerm oil, olive oil, castor oil and sesame oil, glycerol fatty acid esters, polyethylene glycols and fatty acid esters of sorbitan or mixtures of these substances.

In addition to one or more compounds chosen from the compounds of the formulae Ia and Ib, the physiologically compatible salts of the compounds of the formulae Ia and Ib and the stereoisomeric forms of the compounds of the formulae Ia and Ib and optionally in addition to one or more UV filters, suspensions can comprise the customary carrier substances, such as liquid diluents, e.g. water, ethanol or propylene glycol, suspending agents, e.g. ethoxylated isostearyl alcohols, polyoxyethylene sorbitol esters and polyoxy-ethylene sorbitan esters, microcrystalline cellulose, aluminum metahydroxide, bentonite, agar agar and tragacanth or mixtures of these substances.

In addition to one or more compounds chosen from the compounds of the formulae Ia and Ib, the physiologically compatible salts of the compounds of the formulae Ia and Ib and the stereoisomeric forms of the compounds of the formulae Ia and Ib and optionally in addition to one or more UV filters, soaps can comprise the customary carrier substances, such as alkali metal salts of fatty acids, salts of fatty acid half-esters, fatty acid protein hydrolyzates, isothionates [sic], lanolin, fatty alcohol, vegetable oils, plant extracts, glycerol, sugars or mixtures of these substances.

In addition to one or more compounds chosen from the compounds of the formulae Ia and Ib, the physiologically compatible salts of the compounds of the formulae Ia and Ib and the stereoisomeric forms of the compounds of the formulae !a and Ib and optionally in addition to one or more UV filters, surfactant-containing cleansing products can comprise the customary carrier substances, such as salts of fatty alcohol sulfates, fatty alcohol ether sulfates, sulfo-succinic half-esters, fatty acid protein hyrolyzates, isothionates [sic], imidazolinium derivatives, methyltaurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters or mixtures of these substances.

In addition to one or more compounds chosen from the compounds of the formulae Ia and Ib, the physiologically compatible salts of the compounds of the formulae Ia and Ib and the stereoisomeric forms of the compounds of the formulae Ia and Ib and optionally in addition to one or more UV filters, face and body oils can comprise the customary carrier substances, such as synthetic oils, such as fatty acid esters, fatty alcohols, silicone oils, natural oils, such as vegetable oils and oily plant extracts, paraffin oils, lanolin oils or mixtures of these substances.

Further typically cosmetic application forms are also lipsticks, lipcare sticks, mascara, eyeliner, eyeshadows, blusher, powder, emulsion and wax foundation, and sunscreen, presun and aftersun preparations.

The proportion of the compounds chosen from the compounds of the formulae Ia and Ib, the physiologically compatible salts of the compounds of the formulae Ia and Ib, and the stereoisomerc forms of the compounds of the formulae Ia and Ib in the cosmetic formulation is preferably from 0.0001 to 50% by weight, particularly preferably from 0.001 to 10% by weight, based on the total cosmetic formulation.

If UV filters are present in the cosmetic formulation, their proportion is preferably from 0.001 to 50% by weight, particularly preferably from 0.01% by weight to 10% by weight, based on the total cosmetic formulation.

DNA damage of skin cells can be determined, for example, by established and validated tests, e.g. by means of “Comet assay”.

The protection of the DNA against endonucleases or restriction enzymes by ectoin or its derivatives can, for example, be detected in vitro. For this purpose, the DNA is fragmented in a targeted manner by said enzymes. The degree of fragmentation is then determined by means of electrophoresis. A comparison of the results for fragmentation experiments which have firstly been carried out in the presence of ectoin and secondly without ectoin demonstrates the protection of the DNA against endonucleases and restriction enzymes by ectoin or ectoin derivatives.

All compounds or components which can be used in the cosmetic formulations are either known and available commercially or can be synthesized by known methods.

The examples below serve to illustrate the invention and are in no way to be regarded as a limitation. All percentages are percentages by weight.

### EXAMPLE 1

The following components are used to prepare a skincare gel (O/W) according to the invention comprising ectoin:

| | | | | % by wt. |
|---|---|---|---|---|
| A | Almond oil | | (2) | 8.0 |
| | Eutanol G | | (3) | 2.0 |
| | Luvitol EHO | | (4) | 6.0 |
| | Oxynex K liquid | (Art. No. 108324) | (1) | 0.05 |
| B | Panthenol | (Art. No. 501375) | (1) | 0.5 |
| | Karion F liquid | (Art. No. 102993) | (1) | 4.0 |
| | Preservative | | | q.s. |
| | Water, demineralized | | | ad 100 |
| C | Sepigel 305 | | (5) | 3.0 |
| D | Ectoin | | (1) | 1.0 |

Preservatives which may be used are

0.05% of propyl-4-hydroxybenzoate (Art. No. 107427) or

0.15% of methyl 4-hydroxybenzoate (Art. No. 106757).

Preparation:

The combined phase B is slowly introduced into phase C with stirring. The predissolved phase A is then added. The mixture is stirred until the phases are homogeneously mixed. Phase D is then added and stirred until homogeneity is reached.

Sources of Supply:

(1) Merck KOaA, Darmstadt

(2) Gustav Heess, Stuttgart

(3) Henkel KGaA, Düsseldorf

(4) BASF A G, Ludwigshafen

(5) Seppic, France

### EXAMPLE 2

The following components are used to prepare a cream (O/W) according to the invention comprising ectoin:

| | | | | % by wt. |
|---|---|---|---|---|
| A | Paraffin, low- | (Art. No. 107174) | (1) | 8.0 |
| | viscosity | | | |
| | Isopropyl myristate | (Art. No. 822102) | (1) | 4.0 |
| | Mirasil CM 5 | | (2) | 3.0 |
| | Stearic acid | | (1) | 3.0 |
| | Arlacel 165 | | (3) | 5.0 |
| B | Glycerol, 87% | (Art. No. 104091) | (1) | 3.0 |
| | Germaben II | | (4) | 0.5 |
| | Water, demineralized | | | ad 100 |
| C | Bianca perfume | | (5) | 0.3 |
| D | Ectoin | | (1) | 1.0 |

Preparation:

Phases A and B are firstly heated separately to 75° C. Phase A is then slowly added to phase B with stirring, and stirred until a homogeneous mixture forms. Following homogenization of the emulsion, it is cooled to 30° C. with stirring, and phases C and D are added and the mixture is stirred until homogeneity is reached.

Sources of supply:

(1) Merck KGaA, Darmstadt

(2) Rhodia

(3) ICI

(4) ISP

(5) Dragoco

### EXAMPLE 3

The following components are used to prepare a sunscreen lotion (W/O) according to the invention comprising ectoin and the UV filter Eusolex T-2000:

| | | | | % by wt. |
|---|---|---|---|---|
| A | Abil WE 09 | | (2) | 5.0 |
| | Jojoba oil | | (3) | 6.0 |
| | Cetiol V | | (4) | 6.0 |
| | Prisorine 2021 | | (5) | 4.5 |
| | Castor oil | | (6) | 1.0 |
| | Lunacera M | | (7) | 1.8 |
| | Miglyol 812 neutral | | | |
| | oil | | (8) | 4.5 |
| B | Eusolex T-2000 | (Art. No. 105373) | (1) | 3.0 |
| | Glycerol, 87% | (Art. No. 104091) | (1) | 2.0 |
| | Sodium chloride | (Art. No. 106400) | (1) | 0.4 |
| | Preservative | | | q.s. |
| | Water, demineralized | | | ad 100 |
| C | Perfume | | (5) | 0.3 |
| D | Ectoin | | (1) | 1.0 |

Preservatives which may be used are

0.05% of propyl-4-hydroxybenzoate (Art. No. 107427) or

0.15% of methyl 4-hydroxybenzoate (Art. No. 106757).

Preparation:

Firstly, Eusolex T-2000 is stirred into phase B and heated to 80° C. Phase A is then heated to 75° C., and phase B is slowly added with stirring. The mixture is stirred until homogeneity is achieved and then cooled to 30° C. with stirring. Phases C and D are then added and the mixture is stirred until homogeneity is achieved.

Sources of Supply:

(1) Merck KOaA, Darmstadt

(2) Th. Goldschmidt A G, Essen

(3) H. Lamotte, Bremen

(4) Henkel KGaA, Düsseldorf

(5) Unichema, Emmerich

(6) Gustav Heess, Stuttgart

(7) H. B. Fuller, Lüneburg

(8) Hüls Troisdorf A G, Witten

### EXAMPLE 4

An investigation is carried-out to determine whether ectoin has an inhibiting action on the UV-induced formation of “sunburn cells” in organotypical skin equivalents.

The investigations are carried out in accordance with the method described in A. R. Young, ‘The sunburn cell’, Photodermatology 4 (1987) 127-134. The cells used are organotypical skin models. Skinethic® skin equivalents from Skinethic, Nice, France (Lot. No. PK 2 DT 07 99 022A 0603) are used. Ectoin is weighed in directly prior to the start of the test and dissolved in the use concentration of 4% in EBSS (Gibco BRL; Order No. 15015 044). The organotypical skin equivalents (Skinethic) are, depending on the treatment group, incubated for 24 hours with or without ectoin (4%) at 37° C. and 5% CO₂. The two treatment groups are then subjected to UV irradiation (0, 30, 60, 100, 200, 300 mJ/cm² of UVB) using a sun simulator (SOL 500, Dr. Hönle). Following incubation for a further 24 hours at 37° C. and 5% CO₂, all skin equivalents are prepared histologically and investigated for induced “sunburn cells”.

The results are given in Tables 1 and 2 and shown graphically in FIG. 1.

TABLE 1
Number of counted “sunburn cells” in the six
individual sections of the UV-irradiated skin
equivalents (no pretreatment with ectoin)

| | Irradiation [mJ/cm²] | | | | | |
|---|---|---|---|---|---|---|
| Section | 0 | 30 | 60 | 100 | 200 | 300 |
| 1 | 0 | 2 | 9 | 75 | >100 | >100 |
| 2 | 0 | 2 | 14 | 89 | >100 | >100 |
| 3 | 0 | 4 | 16 | 83 | >100 | >100 |
| 4 | 1 | 2 | 12 | 73 | >100 | >100 |
| 5 | 0 | 5 | 14 | 82 | >100 | >100 |
| 6 | 0 | 4 | 18 | 98 | >100 | >100 |
| M | 0.17 | 3.17 | 13.83 | 83.33 | >100 | >100 |
| SD | 0.41 | 1.33 | 3.13 | 9.22 | 0.00 | 0.00 |

M: mean, SD: standard deviation.

TABLE 2
Number of counted “sunburn cells” in the six
individual sections of the UV-irradiated skin
equivalents (pretreatment with ectoin)

| | Irradiation [mJ/cm²] | | | | | |
|---|---|---|---|---|---|---|
| Section | 0 | 30 | 60 | 100 | 200 | 300 |
| 1 | 0 | 0 | 3 | 8 | 32 | >100 |
| 2 | 0 | 2 | 3 | 9 | 34 | >100 |
| 3 | 0 | 1 | 2 | 9 | 39 | >100 |
| 4 | 0 | 1 | 4 | 10 | 32 | >100 |
| 5 | 0 | 2 | 3 | 10 | 39 | <100 |
| 6 | 0 | 3 | 2 | 8 | 35 | >100 |
| M | 0.00 | 1.50 | 2.83 | 9.00 | 35.17 | >100 |
| SD | 0.00 | 1.05 | 0.75 | 0.89 | 3.19 | 0.00 |

M: mean, SD: standard deviation.

FIG. 1 gives the means M of the histologically evaluated sections and also the standard deviation SD from Tab. 1 and Tab. 2.

The histological examination of the two unirradiated control cultures (without ectoin and with preincubation for 24 hours with ectoin) shows that they are living, multilayered epithelia. On the skin equivalents, it could clearly be seen that a heavily structured Stratum corneum lies on this epithelium as the outermost barrier. The heavily granulated cells of the Stratum granulosum equivalent can clearly be seen between the Stratum corneum and the still vital cell layers. In all of the sections of the unirradiated controls analyzed, only one diskeratotic cell was detectable in the lower layers.

The epidermal tissue [sic] which have been irradiated with 30 mJ/cm² of UVB basically have the same morphological structure. However, isolated sunburn cells (SBCs) are already detectable, with there being a tendency for more SBCs to be detectable in the preparation without preconditioning (cf. Tab. 2).

Following irradiation with 60 mJ/cm² of UVB, this tendency continues. Although now isolated SBCs can be detected in the organotypical skin models pretreated with ectoin (2.83±0.75 per section), there are considerably fewer than in the skin model not treated with ectoin (cf. Tab. 2).

In the case of an irradiation dose of 100 mJ/cm² of UVB, the overall morphology is, as before, retained for both test mixtures with and without preincubation for 24 hours with ectoin, although the number of UV-induced SBCs increases drastically in the test mixture without pretreatment with ectoin (83.33±9.22 per section), whereas in the pretreated model the occurrences are still extremely infrequent (9±0.89) (cf. Tab. 2).

Following irradiation with 200 mJ/m² of UVB, in the case of the non-pretreated skin model, damage and SBC formation occur to such a massive extent that exact quantification is no longer possible. The culture pretreated with ectoin now also exhibits significant formation rates of SBCs. However, the number (35.17±3.19) remains significantly below the level of the 100 mJ/cm² UVB mixture without pretreatment (cf. Tab. 2).

Following irradiation with 300 mJ/cm² of UVB, the UV-induced damage for both test mixtures is so great that quantification of SBCs formed is no longer possible. At this chosen high UV dose even partial necrotic tissue damage results.

We were therefore able to show that there is a dose-effect relationship between the rate of formation of SBCs and the administered UV dose. However, this dose-effect relationship could be shifted significantly to higher UV dose ranges as a result of a pretreatment with ectoin.

1. Use of one or more compounds chosen from the compounds of the formulae Ia and Ib 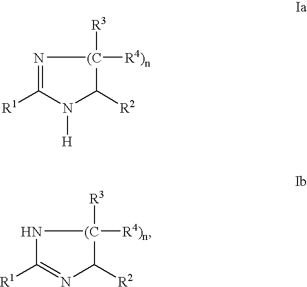
the physiologically compatible salts of the compounds of the formulae Ia and Ib, and the stereoisomeric forms of the compounds of the formulae Ia and Ib, where
R¹ is H or alkyl,
R² is H, COOH, COO-alkyl or CO—NH—R⁵,
R³ and R⁴ in each case independently of one another are H or OH,
n is 1, 2 or 3,
alkyl is an alkyl radical having 1 to 4 carbon atoms, and
R⁵ is H, alkyl, an amino acid radical, dipeptide radical or tripeptide radical for the preparation of a cosmetic formulation for the protection and stabilization of nucleic acids of human skin cells.

2. Use according to claim 1, characterized in that the nucleic acids of human skin cells are protected and stabilized against physical, chemical and biological influences.

3. Use according to claim 1, characterized in that the nucleic acids of human skin cells are protected against UV radiation.

4. Use according to claim 1, characterized in that the nucleic acids of human skin cells are protected and stabilized against denaturing substances.

5. Use according to claim 1, characterized in that the nucleic acids of human skin cells are protected and stabilized against enzymes.

6. Use according to claim 1, characterized in that the nucleic acids of human skin cells are protected and stabilized against viruses.

7. Use according to claim 1, characterized in that the nucleic acids of human skin cells are protected and stabilized against Herpes viruses.

8. Use according to claim 3, characterized in that the cosmetic formulation, in addition to comprising one or more compounds chosen from the compounds of the formulae Ia and Ib, the physiologically compatible salts of the compounds of the formulae Ia and If arid the stereoisomeric forms of the compounds of the formulae Ia and Ib, comprises one or more UV filters.

9. Use according to one of claims 1 to 8, characterized in that the nucleic acids of the cells of the epidermis are protected and stabilized.

10. Use according to one of claims 1 to 9, characterized in that one or more compounds chosen from the compounds of the formulae Ia and Ib, the physiologically compatible salts of the compounds of the formulae Ia and Ib and the stereoisomeric forms of the compounds of the formulae Ia and Ib, optionally together with one or more UV filters is/are used for external application in the form of a solution, a suspension, an emulsion, a paste, an ointment, a gel, a cream, a lotion, a powder, a soap, a surfactant-containing cleansing preparation, an oil, a lipstick, a lipcare stick, a mascara, an eyeliner, eyeshadows, blusher, a powder, emulsion or wax foundation, a sunscreen, presun and aftersun preparation or a spray.

11. Use according to one of claims 1 to 10, characterized in that the proportion of the compounds chosen from the compounds of the formulae Ia and Ib, the physiologically compatible salts of the compounds of the formulae Ia and Ib, and the stereoisomeric forms of the compounds of the formulae Ia and Ib is from 0.0001 to 50% by weight, based on the total cosmetic formulation.

12. Use according to one of claims 1 to 11, characterized in that the compounds of the formulae Ia and Ib are chosen from the compounds (S)-1,4,5,6-tetrahydro-2-methyl-4-pyrimidine-carboxylic acid and (S,S)-1,4,5,6-tetrahydro-5-hydroxy-2-methyl-4-pyrimidinecarboxylic acid.

13. Cosmetic formulation comprising
a) one or more compounds chosen from the compounds of the formulae Ia and Ib 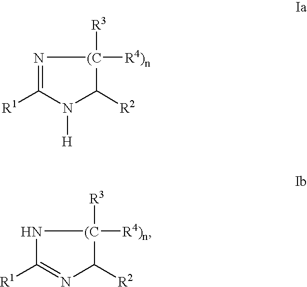
the physiologically compatible salts of the compounds of the formulae Ia and Ib, and the stereoisomeric forms of the compounds of the formulae Ia and Ib, where
R¹ is H or alkyl,
R² is H, COOH, COO-alkyl or CO—NH—R⁵,
R³ and R⁴ in each case independently of one another are H or OH,
n is 1, 2 or 3,
alkyl is an alkyl radical having 1 to-4 carbon atoms, and
R⁵ is H, alkyl, an amino acid radical, dipeptide radical or tripeptide radical and
b) one or more UV filters.

14. Cosmetic formulation according to claim 13, characterized in that the compounds of the formulae Ia and Ib are chosen from the compounds (S)-1,4,5,6-tetrahydro-2-methyl-4-pyrimidine-carboxylic acid and (S,S)-1,4,5,6-tetrahydro-5-hydroxy-2-methyl-4-pyrimidinecarboxylic acid.

15. Cosmetic formulation according to one of claims 13 or 14, characterized in that the UV filters are chosen from organic and inorganic UV filters.

16. Cosmetic formulation according to claim 15, characterized in that the organic UV filters are chosen from benzoyl- or dibenzoylmethane derivatives, methoxycinnamates, salicylate derivatives, benzylidenecamphor derivatives, octocrylene, benzophenones, phenylbenzimidazole-5-sulfonic acid, 4-aminobenzoic acid, octyl triazone and octyl dimethyl PABA.

17. Cosmetic formulation according to one of claims 15 or 16, characterized in that the inorganic UV filters are chosen from titanium dioxide and zinc oxide.

18. Cosmetic formulation according to one of claims 13 to 17, characterized in that the proportion of the compounds chosen from the compounds of the formulae Ia and Ib, the physiologically compatible salts of the compounds of the formulae Ia and Ib, and the stereoisomeric forms of the compounds of the formulae Ia and Ib is from 0.0001,to 50% by weight, based on the total cosmetic formulation, and the proportion of UV filters is from 0.01 to 10% by weight, based on the total cosmetic formulation.

19. Cosmetic formulation according to one of claims 13 to 18, characterized in that it is in the form of a solution, a suspension, an emulsion, a paste, an ointment, a gel, a cream, a lotion, a powder, a soap, a surfactant-containing cleansing preparation, an oil, a lipstick, a lipcare stick, a mascara, an eyeliner, eyeshadows, blusher, a powder, emulsion or wax foundation, a sunscreen, presun and aftersun preparation or a spray.
